(19) United States
(12) Patent Application Publication
(10) Pub. No.: US 2011/0293694 A1
(43) Pub. Date: Dec. 1, 2011

(54) QUANTUM UNIT OF INHERITANCE VECTOR THERAPY METHOD

(76) Inventors: LANE BERNARD SCHEIBER, Annandale, VA (US); LANE BERNARD SCHEIBER, II, Grosse Ile, MI (US)

(21) Appl. No.: 12/790,936

(22) Filed: May 31, 2010

(51) Int. Cl. A61K 9/127; A61P 43/00; A61K 31/711; A61K 9/50; A61K 31/7105

(52) U.S. Cl. 424/450; 424/490; 514/44 R

(57) ABSTRACT

The innovative treatment method described here utilizes configurable microscopic medical payload delivery devices to act as a transport vector to deliver quantum genes to specific type of cells in the body. Utilizing probes on the exterior of the transport device, transport device locate specific target cell types in the body. Once a specific target cell type has been encountered, the configurable microscopic medical payload delivery device inserts its payload of quantum genes into the target cell type. By delivering quantum genes to specific type of cells, genes can be activated or inactivated in those specific type of cells. This method of delivering medically therapeutic quantum genes to specific cells is intended to improve cell function or extend the longevity of cells or neutralize harmful cells that pose a hazard to the body.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2010 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention This invention relates to any medical method intended to correct a protein deficiency or genetic deficiency in the body by utilizing a configurable microscopic medical payload delivery device to insert one or more quantum genes into one or more specific type of cells in the body to improve cell function.

2. Description of Background Art

The Central Dogma of Microbiology dictates that in the nucleus of a cell, genes are transcribed to produce messenger ribonucleic acid molecules (mRNAs), these mRNAs migrate to the cytoplasm where they are translated to produce proteins. One of the great unknowns that has challenged the study of microbiology is the subject of understanding of how the genes, comprising the genome of a species, are organized such that the nuclear transcription machinery can efficiently locate specific transcribable genetic information and instructions that the cell requires to maintain itself, grow and conduct cell replication. Decoding the means as to how the genetic information contained in the nuclear deoxyribonucleic acid (DNA) of a cell is organized, helps to further the efforts to produce an effective gene therapy treatment strategy. Understanding the basis of genetic instruction code information stored in a cell's DNA and utilizing such knowledge of labeling and cataloging of genetic information, makes inserting biologic instruction into the DNA of cells a practical and effective means of treating a wide scope of medical conditions.

The human genome is comprised of deoxyribonucleic acid (DNA) separated into 46 chromosomes. The chromosomes are further subdivided into genes. Genes represent units of transcribable DNA. Transcription of the DNA refers to generating one or more of a variety of RNA molecules. Regarding the human genome, currently it is estimated that 5% of the total nuclear DNA is thought to represent genes and 95% is thought to represent redundant non-gene genetic material. The DNA genome in a cell is therefore comprised of transcribable genetic information and nontranscribable genetic information. Transcribable genetic information represent the segments of DNA that when transcribed by transcription machinery yield RNA molecules, usually in a precursor form that require modification before the RNA molecules are capable of being translated. The nontranscribable genetic information represent segments that act as either points of attachment for the transcription machinery or act as commands to direct the transcription machinery or act as spacers between transcribable segments of genetic information or have no known function at this time. A segment of nontranslatable DNA that is coded as a STOP command, under the proper circumstances, will cause the transcription machinery to cease transcribing the DNA at that point. A segment of DNA coded to signal a REPEAT command, will cause the transcription machinery to repeat its transcription of a segment of genetic information. The term ‘genetic information’ refers to a sequence of nucleotides that comprise transcribable portions of DNA and nontranscribable portions of DNA. In the DNA, four different nucleotides comprise the nucleotide sequences. The four different nucleotides that comprise the DNA include adenine, cytosine, guanine, and thymine.

Computer programs, commonly utilized in desk top computers, laptop computers, mainframe computers are comprised of a series of software instructions and data. In order for a computer program to run its digital programming in an orderly fashion, each software instruction and each element of data is assigned or associated with a unique identifier such that the software instructions can be carried out in an orderly fashion and each element of data can be efficiently located when there is a need to process the data elements. Similarly, each unit of genetic information, often referred to as a gene, comprising the nuclear DNA of a species genome, must have a unique identifier assigned to it such that the genetic information can be readily located by the transcription machinery and utilized when needed by a cell.

When a gene is to be transcribed, approximately forty proteins assemble together into what is referred to as a transcription complex, which acts as the transcription machinery. The transcription complex forms along a segment of DNA, upstream from the start of the transcribable genetic information. The transcription complex transcribes the genetic information to produce RNA. It is vital to the cell that the transcription complex is able to locate a specific gene amongst the 3 billion base pairs comprising the human genome in an orderly and efficient fashion to enable it to perform functions the cell requires to operate, survive, grow and replicate.

For purposes of this text there are several general definitions. A ‘ribose’ is a five carbon or pentose sugar (C₅H₁₀O₅) present in the structural components of ribonucleic acid, riboflavin, and other nucleotides and nucleosides. A ‘deoxyribose’ is a deoxypentose (C₅H₁₀O₄) found in deoxyribonucleic acid. A ‘nucleoside’ is a compound of a sugar usually ribose or deoxyribose with a nitrogenous base by way of an N-glycosyl link. A ‘nucleotide’ is a single unit of a nucleic acid, composed of a five carbon sugar (either a ribose or a deoxyribose), a nitrogenous base and a phosphate group. There are two families of ‘nitrogenous bases’, which include: pyrimidine and purine. A ‘pyrimidine’ is a six member ring made up of carbon and nitrogen atoms; the members of the pyrimidine family include: cytosine (C), thymine (T) and uracil (U). A ‘purine’ is a five-member ring fused to a pyrimidine type ring; the members of the purine family include: adenine (A) and guanine (G). A ‘nucleic acid’ is a polynucleotide which is a biologic molecule such as ribonucleic acid or deoxyribonucleic acid that allow organisms to reproduce. A ‘ribonucleic acid’ (RNA) is a linear polymer of nucleotides formed by repeated riboses linked by phosphodiester bonds between the 3-hydroxyl group of one and the 5-hydroxyl group of the next; RNAs are a single strand macromolecule comprised of a sequence of nucleotides, these nucleotides are generally referred to by their nitrogenous bases, which include: adenine, cytosine, guanine or uracil. The term macromolecule refers to any very large molecule. RNAs are subset into different types which include messenger RNA (mRNA), transport RNA (tRNA), ribosomal RNA (rRNA) and a variety of small RNAs. Messenger RNAs act as templates to produce proteins. A ribosome is a complex comprised of rRNAs and proteins and is responsible for the correct positioning of mRNA and charged tRNA to facilitate the proper alignment and bonding of amino acids into a strand to produce a protein. A ‘charged’ tRNA is a tRNA that is carrying an amino acid. Ribosomal RNA (rRNA) represents a subset of RNAs that form part of the physical structure of a ribosome. Small RNAs include snoRNA, U snRNA, and miRNA. The snoRNAs modify precursor rRNA molecules. U snRNAs modify precursor mRNA molecules. The miRNA molecules modify the function of mRNA molecules.

A ‘deoxyribose’ is a deoxypentose (C₅H₁₀O₄) sugar. Deoxyribonucleic acid (DNA) is comprised of three basic elements: a deoxyribose sugar, a phosphate group and nitrogen containing bases. DNA is a macromolecule made up of two chains of repeating deoxyribose sugars linked by phosphodiester bonds between the 3-hydroxyl group of one and the 5-hydroxyl group of the next; the two chains are held antiparallel to each other by weak hydrogen bonds. DNA strands contain a sequence of nucleotides, which include: adenine, cytosine, guanine and thymine. Adenine is always paired with thymine of the opposite strand, and guanine is always paired with cytosine of the opposite strand; one side or strand of a DNA macromolecule is the mirror image of the opposite strand. Nuclear DNA is regarded as the medium for storing the master plan of hereditary information.

Genes are considered segments of the DNA that represent units of inheritance.

A chromosome exists in the nucleus of a cell and consists of a DNA double helix bearing a linear sequence of genes, coiled and recoiled around aggregated proteins, termed histones. The number of chromosomes varies from species to species. Most Human cells carries twenty two pairs of chromosomes plus two sex chromosomes; two ‘x’ chromosomes in women and one ‘x’ and one ‘y’ chromosome in men. Chromosomes carry genetic information in the form of units which are referred to as genes. The entire nuclear genome, forty six chromosomes, is comprised of 3 billion base pairs of nucleotides.

Mitochondria possess numerous circular DNA. The limited information stored in mitochondrial DNA is thought to assist the mitochondria in producing the enzymes needed to convert glucose to adenosine triphosphate.

Various standard definitions of a gene exist. Per Stedman's Medical Dictionary, 24^{th} edition, copyright 1982: The functional unit of heredity. Each gene occupies a specific place or locus on a chromosome, is capable of reproducing itself exactly at cell division, and is capable of directing the formation of an enzyme or other protein. The gene as a functional unit probably consists of a discrete segment of purine (adenine and guanine) and pyrimidine (cytosine and thymine) bases in the correct sequence to code the sequence of amino acids needed to form a specific peptide. Protein synthesis is mediated by molecules of messenger RNA formed on the chromosome with the gene unit of DNA acting as a template, which then pass into the cytoplasm and become oriented on the ribosomes where they in turn act as templates to organize a chain of amino acids to form a peptide. Genes normally occur in pairs in all cells except gametes as a consequence of the fact that all chromosomes are paired except the sex chromosomes (x and y) of the male.’

Per Dorland's Pocket Medical Dictionary, 23^{rd} edition, copyright 1982 the definition of ‘gene’ is ‘the biologic unit of heredity, self-producing, and located at a definite position (locus) on a particular chromosome.’

Per the text Understanding Biology, Second Edition, Peter Raven, George Johnson, Mosby, copyright 1991: ‘Gene: The basic unit of heredity. A sequence of DNA nucleotides on a chromosome that encodes a polypeptide or RNA molecule and so determines the nature of an individual's inherited traits.’

Per The New Oxford American Dictionary, Second Edition, copyright 2005: ‘Gene: A unit of heredity that is transferred from a parent to offspring and is held to determine some characteristic of the offspring: proteins coded directly by genes. In technical use: a distinct sequence of nucleotides forming part of a chromosome, the order of which determines the order of monomers in a polypeptide or nucleic acid molecule which a cell (or virus) may synthesize.’

Per MedicineNet.com (Current as of the time of this publication): According to the official Guidelines for Human Gene Nomenclature, a ‘gene’ is defined as “a DNA segment that contributes to phenotype/function. In the absence of demonstrated function a gene may be characterized by sequence, transcription or homology.” DNA: Genes are composed of DNA, a molecule in the memorable shape of a double helix, a spiral ladder. Each rung of the spiral ladder consists of two paired chemicals called bases. There are four types of bases. They are adenine (A), thymine (T), cytosine (C), and guanine (G). As indicated, each base is symbolized by the first letter of its name: A, T, C, and G. Certain bases always pair together (AT and GC). Different sequences of base pairs form coded messages. The gene: A gene is a sequence (a string) of bases. It is made up of combinations of A, T, C, and G. These unique combinations determine the gene's function, much as letters join together to form words. Each person has thousands of genes—billions of base pairs of DNA or bits of information repeated in the nuclei of human cells—which determine individual characteristics (genetic traits).’

Per Wikipedia.com, referenced to: Group of the Sequence Ontology consortium, coordinated by K. Eilbeck, cited in H. Pearson. (2006). Genetics: what is a gene? Nature, 441, 398-401 (Current as of the time of this publication): A modern working definition of a gene is ‘a locatable region of genomic sequence, corresponding to a unit of inheritance, which is associated with regulatory regions, transcribed regions, and or other functional sequence regions.’

The above definitions of a ‘gene’ are fairly detailed and at present time generally universally accepted in the science and medical communities as representing the definition of a gene. There is a distinct lack of any previous reference in the medical science literature to a unique identifier associated with genetic material.

Current gene theory is derived from Gregor Mendel (1822-1884), who discovered the basic principles of heredity by breeding garden peas at the abbey where he resided, while teaching at Brunn Modern School. Gregor Mendel built and documented a model of inheritance, often referred to as Mendelian genetics, that has acted as the foundation of modern genetics. Gregor Mendel documented changes in characteristics of the plants he grew and described the physical traits as being related to ‘heritable factors’. Over time Mendel's term ‘heritable factor’ has been replaced by the terms ‘gene’ and ‘allele’. Much of what the current term of a ‘gene’ describes remains related to and distinctly linked to the physical traits of the live organisms they describe.

Per J. K. Pal, S. S. Ghaskabi, Fundamentals of Molecular Biology, 2009: ‘The central dogma of molecular biology . . . states that the genes present in the genome (DNA) are transcribed into mRNAs, which are then translated into polypeptides or proteins, which are phenotypes.’ ‘Genome, thus, contains the complete set of hereditary information for any organism and is functionally divided into small parts referred to as genes. Each gene is a sequence of nucleotides representing a single protein or RNA. Genome of a living organism may contain as few as 500 genes as in case of Mycoplasma, or as many as 30,000 genes as in case of human beings.’

Current computer technology utilizes the binary numeric language. Every task a computer performs is related to the language of ‘one’ and ‘zeros’. Transistors that comprise the inside of computer chips are either turned ‘on’ representing a ‘one’ or turned ‘off’ representing a ‘zero’. At the core of all computer programs is the machine language of ‘ones’ and ‘zeros’. The most sophisticated central processing unit (CPU) in the world only reads and processes the language of ‘ones’ and ‘zeros’. All text, all pictures, all video, all sound and music is diluted down to the form of one's and zero's, and consequently all of the computing and storage power of a computer is performed by the computer language of ‘ones’ and ‘zeros’.

The nucleus of a biologically active cell arguably possesses the most sophisticated and well organized processing power in the world. To run such a powerful processing unit, a form of biologic computer language would seem to be a necessary foundation by which to transfer stored information from the DNA to the remainder of the biologically active portions of a cell as needed. Given that the DNA comprising the chromosomes and mitochondrial DNA are both comprised of four different nucleotides including adenosine, cytosine, guanine and thymine, and RNA is comprised of four nucleotides including adenosine, cytosine, guanine and uracil (uracil in place of thymine), it appears evident the biologic computer language used by a cell's genome is an information language derived from base-four mathematics. Instead of current computer technology utilizing binary computer code comprised of ‘ones’ and ‘zeros’, the DNA and RNA in a biologically active cell utilize an information language comprised of ‘zeros’, ‘one's’, two's’ and ‘threes’ to store and transfer information, which in effect represents a base-four language or quaternary language.

The above definitions of a ‘gene’ refer to genes residing in a specific place or locus on a chromosome. Identifying that a gene is present in a particular location is obvious to the human observer, but from a functional standpoint for cell biology this does not necessarily help a cell find or use the information stored in the nucleotide sequence of a particular gene. To rely on location alone, as a means of identifying a gene, would put the function of the entire genome at peril of failure if even a single base pair of nucleotides were added or deleted from the genome. To this point, no discussion regarding genes being organized utilizing a coding system of any form within the genome, other than the mention of physical location in a chromosome, has been made in the medical literature.

The current understanding of the actual biologic structure of a gene is far more elaborate than the standard definition of a gene leads a casual reader to believe; this knowledge has evolved greatly since Gregor Mendel's work in the 19^{th} century. A gene appears to be comprised of a number of segments loosely strung together along a particular section of DNA. In general there are at least three global segments associated with a gene which include: (1) the Upstream 5′ flanking region, (2) the transcriptional unit and (3) the Downstream 3′ flanking region.

The Upstream 5′ flanking region is comprised of the ‘enhancer region’, the ‘promoter-proximal region’, and ‘promoter region’.

The ‘transcriptional unit’ begins at a location designated ‘transcription start site’ (TSS), which is located in a site called the ‘initiator region’ (inR), which may be described in a general form as Py₂CAPy₅. The transcription unit is comprised of the combination of segments of DNA nucleotides to be transcribed into RNA and spacing units known as ‘introns’ that are not transcribed or if transcribed are later removed post transcription, such that they do not appear in the final RNA molecule. In the case of a gene coding for a mRNA molecule, the transcription unit will contain all three elements of the mRNA, which includes: (1) the 5′ noncoding region, (2) the translational region and (3) the 3′ noncoding region. Interspersed between these regions are exons, which will not be transcribed and introns that if transcribed, are removed from the precursor form of mRNA prior to the mRNA reaching its final form. Exons and introns appear to be likened to spacers. The exact role exons and introns play in the transcription process is undetermined.

The Downstream 3′ flanking region contains DNA nucleotides that are not transcribed and may contain what has been termed an ‘enhancer region’. An enhancer region in the Downstream 3′ flanking region may promote the gene previously transcribed to be transcribed again.

On either side of the DNA sequencing comprising a gene and its flanking regions, may be inactive DNA which act as boundaries which have been termed ‘insulator elements’. The term ‘upstream’ refers to DNA sequencing that occurs prior to the TSS if viewed from the 5′ end to the 3′ end of the DNA; where the term ‘downstream’ refers to DNA sequencing located after the TSS.

The ‘enhancer region’ may or may not be present in the Upstream 5′ flanking region. If present in the Upstream 5′ flanking region, the enhancer region helps facilitate the reading of the gene by encouraging formation of the transcription mechanism. An enhancer may be 50 to 1500 base pairs in length occupying a position upstream from the transcription starting site.

The ‘transcription mechanism’, also referred to as the ‘transcription machinery’ or the ‘transcription complex’ (TC), in humans, is reported to be comprised of over forty separate proteins that assemble together to ultimately function in a concerted effort to transcribe the nucleotide sequence of the DNA into RNA. The transcription mechanism includes elements such as ‘general transcription factor Sp1’, ‘general transcription factor NF1’, ‘general transcription factor TATA-binding protein’, ‘TF_{II}D’, ‘basal transcription complex’, and a ‘RNA polymerase protein’ to name only a few of the forty elements that exist. The elements of the transcription mechanism function as (1) a means to recognize the location of the start of a gene, (2) as proteins to bind the transcription mechanism to the DNA such that transcription may occur or (3) as means of transcribing the DNA nucleotide coding to produce a RNA molecule or a precursor RNA molecule.

There are at least three RNA polymerase proteins which include: RNA polymerase I, RNA polymerase II, and RNA polymerase III. RNA polymerase I tends to be dedicated to transcribing genetic information that will result in the formation of rRNA molecules. RNA polymerase II tends to be dedicated to transcribing genetic information that will result in the formation of mRNA molecules. RNA polymerase III appears to be dedicated to transcribing genetic information that results in the formation of tRNAs, small cellular RNAs and viral RNAs.

The ‘promoter proximal region’ is located upstream from the TSS and upstream from the core promoter region. The ‘promoter proximal region’ includes two sub-regions termed the GC box and the CAAT box. The ‘GC box’ appears to be a segment rich in guanine-cytosine nucleotide sequences. The GC box binds to the ‘general transcription factor Sp1’ of the transcription mechanism. The ‘CAAT box’ is a segment which contains the nucleotide sequence ‘GGCCAATCT’ located approximately 75 base pairs (bps) upstream from the transcription start site (TSS). The CAAT box binds to the ‘general transcription factor NF1’ of the transcription mechanism.

The ‘core promoter’ region is considered the shortest sequence within which RNA polymerase II can initiate transcription of a gene The core promoter may include the inR and either a TATA box or a ‘downstream promoter element’ (DPE). The inR is the region designated Py₂CAPy₅ that surrounds the transcription start site (TSS). The TATA box is located 25 base pairs (bps) upstream from the TSS. The TATA box acts as a site of attachment of the TF_{II}D, which is a promoter for binding of the RNA polymerase II molecule. The DPE may appear 28 bps to 32 bps downstream from the TSS. The DPE acts as an alternative site of attachment for the TF_{II}D when the TATA box is not present.

The transcription mechanism or transcription complex appears to be comprised of different elements depending upon whether rRNA is being transcribed versus mRNA or tRNA or small cellular RNA or viral RNA. The proteins that assemble to assist RNA Polymerase I with transcribing the DNA to produce rRNA appear different the proteins that assemble to assist RNA polymerase II with transcribing the DNA to produce mRNA and from the proteins that assemble to assist RNA polymerase III with transcribing the DNA to produce tRNA, small cellular RNA or viral RNA. A common protein that appears to be present at the initial binding of all three types of RNA polymerase molecules is TATA-binding protein (TBP). TBP appears to be required to attach to the DNA, which then facilitates RNA polymerase to bind to the promoter along the DNA. TBP assembles with TBP-associated factors (TAFs). Together TBF and 11 TAFs comprise the complex referred to as TF_{II}D, which has been previously mentioned in the above text.

Upstream from the TATA box is the ‘initiator element’, which may be considered as part of the ‘core promoter’ region. The initiator element is a segment of the nuclear DNA that binds the basal transcription complex. The basal transcription complex is comprised of a number of proteins that make initial contact with the DNA prior to the RNA polymerase binding to the transcription mechanism. The basal transcription complex is associated with an activator.

An activator is a protein comprised of three components. The three components of the activator include: (1) DNA binding domain, (2) Connecting domain, and (3) Activating domain. When the activator's DNA binding domain attaches to the DNA at a specific point along the DNA, the activator's activating domain then causes the other elements of the transcription mechanism to assemble at this location. Generally the assembly of the other proteins occurs downstream from where the activator's DNA binding domain attached to the DNA. There is evidence that the activator is associated with the activity of small RNAs.

The design of the cell is so complex, all of its functions so diverse and intricate that some form of practical order is necessitated. The genes must be ordered in some fashion, especially in a human, where there are at least 30,000 different genes used by the cells. Some estimates place the total number of genes present in the human nuclear DNA genome closer to 100,000. If no means of order existed as to how the genes could be identified, then ‘random circumstance’ would dictate a cell locating a particular portion of genetic information that it requires, at any given time. Randomness tends to favor the occurrence of random events rather than a purposeful order. A ‘random circumstance’ approach to any living cell would tend to favor failure of the cell rather than survival of the cell.

To allow a cell to utilize the biologic information stored in a gene a ‘unique identifier’ (UI) needs to be somehow attached to the gene's specific nucleotide sequence. In the human genome, the cell's transcription mechanisms require an organized means to locate and transcribe any given gene's nucleotide sequence amongst the 3 billion nucleotides that reside in the 46 chromosomes that comprise human DNA. Given how the transcription mechanism assembles upstream from the portion of the gene to be transcribed, the nucleotide sequence acting as a unique identifier associated with a specific gene would be positioned upstream from the transcription start site.

The transcription complex (TC) engages the DNA upstream from the genetic information segment the TC transcribes. The unique identifier may be attached directly to the RNA coding segment of genetic material, or there may exist one or more base pairs physically separating the unique identifier and the RNA coding portion of genetic material. Regarding some genes, there may be numerous base pairs separating the unique identifier from the transcribing region of the gene.

For any form of ‘gene therapy’ to work efficiently, medically therapeutic genetic material inserted into the native DNA of a cell needs to be associated with a unique identification. Attaching a unique identifier to medically therapeutic genetic material is essential in making it possible for the components of a transcription complex to, in a timely organized fashion, locate the exogenous medically therapeutic genetic material, assemble around this exogenous genetic material, and decode the information contained therein. If no such unique identifier is used, then utilization of such exogenous transcribable genetic information occurs based on the occurrence of random events rather than dictated by therapeutic design.

Naturally occurring unique identifiers in the nuclear genome may occur in numerous forms. Since humans share 47% of their DNA with a banana and 95% of their DNA with a monkey, a portion of the unique identifiers associated with genes in the nuclear DNA may not be specific to a human. Unique identifiers may have a global utility, with a portion of the genome of any organism being shared amongst numerous species. The rational would be that once Nature developed an adequate fundamental design for a particular facet of biologic organisms, this information may be shared amongst numerous species that would benefit from the design. An example might be the basic design of a eukaryote cell; this information would be shared amongst all life that utilized the eukaryote cell design rather than each successive multi-celled species having to repeatedly re-invent the design of a eukaryote cell.

In order for the knowledge base of cellular genetics to progress forward, the definition of a gene must be expanded to include the presence of a ‘unique identifier’ associated with each gene present within the DNA. The basis for the presence of this unique identifier (UI) associated with each active gene is so that the cell can locate the biologic information stored in the DNA nucleotide sequencing of the gene. An active gene refers to those genes present in the genome that are utilized by a particular species to support conception, development and maintenance of a species.

Upon adding a unique identifier to a gene, the current term ‘gene’ is thus expanded to the term ‘quantum gene’. The term ‘quantal’ in biology generally refers to an ‘all or nothing’ state or response. The term ‘quantal’ is a derivative of the word quantum. The term ‘quantum means a quantity or amount, and a discrete quantity of energy or a discrete bundle of energy or a discrete quantity of electromagnetic radiation’.

A ‘quantum gene’ is comprised of a sequence of nucleotides that represents a ‘unique identifier’ physically linked to a sequence of nucleotides that represent a discrete quantity of genetic information; these sequences of nucleotides being comprised of some combination of the nucleotides being referred to by their nitrogenous base as adenine (A), thymine (T), cytosine (C), and guanine (G). The genetic information associated with the above-mentioned unique identifier may be comprised of a portion of transcribable genetic information and a portion of nontranscribable genetic information which together define a specific gene, otherwise referred to as a discrete quantity of genetic information.

Similar to how a gene is described, with regards to a quantum gene, the term ‘upstream’ refers to DNA sequencing that occurs prior to the transcription start site (TSS) if viewed from the 5′ end to the 3′ end of the DNA; where the term ‘downstream’ refers to DNA sequencing located after the TSS.

Similar to the previously described organization of a standard gene found in nuclear DNA, a quantum gene is structured with at least three global segments which include: (1) the Upstream 5′ flanking region, (2) the transcriptional unit and possibly instructional units and (3) the Downstream 3′ flanking region. The ‘unique identifier’ is located in the Upstream 5′ flanking region. The current standard definition of a gene strictly encompasses the concept that a gene is comprised of a segment of nuclear DNA that when transcribed produces RNA. Therefore, the differences between the current standard definition of a ‘gene’ and the definition of a ‘quantum gene’ is that a quantum gene includes both a unique identifier and a segment of nuclear DNA that when transcribed produces RNA. The segment of nuclear DNA that when transcribed produces RNA is comprised of one or more segments of transcribable genetic information that may be accompanied by one or more segments of nontranscribable genetic information. Nontranscribable segments of genetic information include segments that are removed or ignored during the transcription process or segments that act as commands which includes a START code, STOP code or a REPEAT code.

When present, a START code signals initiation of the transcription process. When present, a STOP code signals the discontinuation of the transcription process. When present, a REPEAT code signals that the transcription process should repeat the transcription of the segment of DNA that was just transcribed.

Similar to the standard description of a ‘gene’, a quantum gene's Upstream 5′ flanking region is comprised of the ‘enhancer region’, the ‘promoter-proximal region’, and ‘promoter region’.

Similar to the standard description of a ‘gene’, a quantum gene's ‘transcriptional unit’ begins at a location designated ‘transcription start site’ (TSS), which is located in a site called the ‘initiator region’ (inR), which may be described in a general form as Py₂CAPy₅. The transcription unit is comprised of the combination of segments of DNA nucleotides to be transcribed into RNA and spacing units known as ‘exons’ AND ‘introns’, whereby exons represent segments that are not transcribed and introns represent segments that are transcribed but later removed post transcription, such that they do not appear in the final RNA molecule. In the case of a gene coding for a mRNA molecule, the transcription unit will contain all three elements of the mRNA, which includes: (1) the 5′ noncoding region, (2) the translational region and (3) the 3′ noncoding region. Interspersed between these regions are exons, which will not be transcribed and introns that if transcribed, are removed from the precursor form of mRNA prior to the mRNA reaching its final form. Exons and introns present in nuclear DNA appear to be likened to spacers interspersed in the nuclear DNA. The exact role exons and introns play in the transcription process is undetermined.

Similar to the standard description of a ‘gene’, the quantum gene's Downstream 3′ flanking region contains DNA nucleotides that are not transcribed and may contain what has been termed an ‘enhancer region’. An enhancer region in the Downstream 3′ flanking region may promote the gene previously transcribed to be transcribed again.

On either side of the DNA sequencing comprising a gene and a quantum gene are flanking regions which represent inactive DNA, which act as boundaries which have been termed ‘insulator elements’. Insulator elements are areas that are not transcribed to produce RNA. The function of insulator elements, other than acting as boundary markers between differing genes, is unknown at this time.

In nuclear DNA, quantum genes are comprised of a segment of deoxyribonucleic acid where the portion that represents a unique identifier may be separated from the portion that represents transcribable genetic information by a quantity of base pairs of nucleotides that do not represent a unique identifier and do not represent transcribable genetic information. The purpose of the separation of the portion of the unique identifier from the portion of the genetic information by a quantity of base pairs of nucleotides that do not represent a unique identifier and does not represent genetic information may be to act to facilitate a transcription complex attaching to the quantum gene upstream from the portion of the quantum gene that represents genetic information so that transcription of the biologic information associated with the quantum gene may occur at the designated starting point.

The unique identification or identifier of a quantum gene could be in the form of nucleotide sequence that represents a name assigned to the quantum gene, or a number assigned to a quantum gene or the combination of a name and number assigned to a quantum gene. Irrespective of whether the unique identifier incorporated in a quantum gene is considered a ‘name’, or a ‘number’ or a combination of a name or number, the unique identifier is comprised of a sequence of nucleotides linked to the transcribable genetic information for which it acts as a unique identifier. It has been estimated that there are as many as 100,000 separate genes stored in the DNA of the 46 chromosomes comprising the human genome. In a base four language, a string of nine nucleotides is needed to code for 256,144 individual genes. If there were over a million quantum genes, then a string of ten nucleotides could be used since ten nucleotides could represent 1,024,576 unique numbers in a base-four number system.

Utilizing a base four number system a string of twenty-five nucleotides would represent the number 1,125,899,906,842,624, which could account for 200,000 different quantum genes in 5 billion different species. Therefore 200,000 different quantum genes could be dedicated to producing a biped form of life. In the human genome 5% of the 3 billion base pairs are considered to represent genes by the current definition of a gene. If 5% of the human genome represents the 100,000 quantum genes in the nuclear DNA, then on average 1500 nucleotides can be dedicated to each gene. If 25 nucleotides are dedicated to a unique address or unique identifier, then there remain 1475 nucleotides, on average, to be utilized for coding the biologic information associated with each of the 100,000 quantum genes estimated to exist in the human genome.

A unique identifier (UI) incorporated in quantum genes could be comprised of a unique number or a unique name or the unique combination of a number and a name. A name might be represented as a single letter or a series of letters. The current convention utilized in science is to apply the four letter alphabet A, C, G, T to represent the four different bases of the nucleotides comprising the DNA, which include adenosine, cytosine, guanine, and thymine respectfully. With regards to RNA, the four letter alphabet A, C, G, U is utilized to represent the bases of the nucleotides which include adenosine, cytosine, guanine, and uracil. Regarding utilizing a unique identifier for DNA, a name could be comprised of a series of letters derived from the four letters A, C, G, and T. Regarding utilizing a unique identifier for purposes of use within an RNA molecule, a unique identifier could be comprised of a series of letters derived from the four letters A, C, G, U. The current scientific convention does not recognize a mathematical base-four nomenclature regarding DNA or RNA. The unique identifier could be represented as a number. Names can be translated into numbers and vice versa.

In the nuclear DNA, there are several places in the upstream segment of a quantum gene where a segment of twenty-five or more base pairs could exist that acts as the unique identifying code that uniquely identifies the segment of transcribable genetic information. The transcription start site (TSS) is present upstream from a segment of transcribable genetic information. There exists a segment of 25 bps upstream from the TSS that occupies the space along the DNA between the TSS and the TATA box. There exists the downstream promoter element (DPE) 28 bps to 32 bps downstream from the TSS. The DPE acts as an alternative site of attachment for the TF_{II}D when the TATA box is not present. Within the 28 bps to 32 bps of DNA separating the DPE from the TSS may also be a convenient location for a unique identifying code to reside and be associated with the genetic information located just downstream. The cell exists with numerous variability. There exists variation in the arrangement of the elements upstream from the transcribable genetic information, therefore various sites upstream from the transcribable genetic information may function as the unique identifying code for some quantum genes. The unique identifying code may be represented as subsegments of DNA, where subsegments are physically separated from each other, but in combination, the subsegments act in unison to identify a segment of transcribable genetic information.

By delivering quantum genes containing the genetic information required to produce insulin directly to the cells responsible for the production of insulin, the medical treatment of diabetes mellitus is significantly improved. Diabetes mellitus represents a state of hyperglycemia, a serum blood sugar that is higher than what is considered the normal range for humans. Glucose, a six-carbon molecule, is a form of sugar. Glucose is absorbed by the cells of the body and converted to energy by the processes of glycolysis, the Krebs cycle and phosporylation. Insulin, a protein, facilitates the transfer of glucose from the blood into cells. Normal range for blood glucose in humans is generally defined as a fasting blood plasma glucose level of between 70 to 110 mg/dl. For descriptive purposes, the term ‘plasma’ refers to the fluid portion of blood.

Diabetes mellitus is classified as Type One and Type Two. Type One diabetes mellitus is insulin dependent, which refers to the condition where there is a lack of sufficient insulin circulating in the blood stream and insulin must be provided to the body in order to properly regulate the blood glucose level. When insulin is required to regulate the blood glucose level in the body, this condition is often referred to as insulin dependent diabetes mellitus (IDDM). Type Two diabetes mellitus is noninsulin dependent, often referred to as noninsulin dependent diabetes mellitus (NIDDM), meaning the blood glucose level can be managed without insulin, and instead by means of diet, exercise or intervention with oral medications. Type Two diabetes mellitus is considered a progressive disease, the underlying pathogenic mechanisms including pancreatic Beta cell (also often designated as β-Cell) dysfunction and insulin resistance.

The pancreas serves as an endocrine gland and an exocrine gland. Functioning as an endocrine gland the pancreas produces and secretes hormones including insulin and glucagon. Insulin acts to reduce levels of glucose circulating in the blood. Beta cells secrete insulin into the blood when a higher than normal level of glucose is detected in the serum. For purposes of this description the terms ‘blood’, ‘blood stream’ and ‘serum’ refer to the same substance. Glucagon acts to stimulate an increase in glucose circulating in the blood. Beta cells in the pancreas secrete glucagon when a low level of glucose is detected in the serum.

Glucose enters the body and then the blood stream as a result of the digestion of food. The Beta cells of the Islets of Langerhans continuously sense the level of glucose in the blood and respond to elevated levels of blood glucose by secreting insulin into the blood. Beta cells produce the protein ‘insulin’ in their endoplasmic reticulum and store the insulin in vacuoles until it is needed. When Beta cells detect an increase in the glucose level in the blood, Beta cells release insulin into the blood from the described storage vacuoles.

Insulin is a protein. An insulin protein consists of two chains of amino acids, an alpha chain and a beta chain, linked by two disulfide (S—S) bridges. One chain, the alpha chain consists of 21 amino acids. The second chain the beta chain consists of 30 amino acids.

Insulin interacts with the cells of the body by means of a cell-surface receptor termed the ‘insulin receptor’ located on the exterior of a cell's ‘outer membrane’, otherwise known as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood and convert it to glycogen to facilitate the cells in the body to utilize glucose to generate biochemically usable energy, and to stimulate fat cells to take up glucose and synthesize fat.

Diabetes Mellitus may be the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the deoxyribonucleic acid (DNA) to create messenger ribonucleic acid (mRNA) molecules, which facilitate the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the Beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

A ‘eukaryote’ refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure receptors that the cell uses to detect properties of the environment surrounding the cell membrane. The cytoplasm acts as a filling medium inside the boundaries of the plasma cell membrane and is comprised mainly of water and nutrients such as amino acids, oxygen, and glucose. The nucleus, organelles, and ribosomes are suspended in the cytoplasm. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex (sometimes referred to as a Golgi apparatus). Floating in the cytoplasm, but also located in the endoplasmic reticulum and mitochondria are ribosomes. Ribosomes are complex macromolecule structures comprised of ribosomal ribonucleic acid (rRNA) molecules and ribosomal proteins that combine and couple to a messenger ribonucleic acid (mRNA) molecule. The rRNAs and the ribosomal proteins congregate to form a macromolecule structure that surrounds a mRNA molecule. Ribosomes decode genetic information in a mRNA molecule and manufacture proteins to the specifications of the instruction code physically present in the mRNA molecule. More than one ribosome may be attached to a single mRNA at a time.

Proteins are comprised of a series of amino acids bonded together in a linear strand, sometimes referred to as a chain; a protein may be further modified to be a structure comprised of one or more similar or differing strands of amino acids bonded together. Insulin is a protein structure comprised of two strands of amino acids; one strand comprised of 21 amino acids long and the second strand comprised of 30 amino acids, the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors. Messenger RNAs (mRNA) are created by transcription of DNA, they generally migrate to other locations inside the cell and are utilized by ribosomes as protein manufacturing templates. A ribosome is a protein complex that manufactures proteins by deciphering the instruction code located in a mRNA molecule. When a specific protein is needed, pieces of the ribosome complex, which include rRNA molecules and ribosomal proteins, bind around the strand of a mRNA that carries the specific instruction code that will generate the required protein. The ribosome traverses the mRNA strand and deciphers the genetic information coded into the sequence of nucleotides that comprise the mRNA molecule to produce a protein molecule and this process is referred to as translation.

The insulin molecule is a protein produced by Beta cells located in the pancreas. The ‘insulin messenger RNA’ is created in a Beta cell by a polymerase complex transcribing the insulin gene from nuclear DNA in the nucleus of the cell. The native messenger RNA (mRNA) for insulin then travels to the endoplasmic reticulum, where numerous ribosomes, comprised of rRNA and ribosomal proteins, engage these mRNA molecules. Many ribosomes may be attached to a single strand of mRNA simultaneously, each generating an identical copy of the protein as dictated by the information encoded in the mRNA. Insulin is produced by ribosomes translating the information in a mRNA molecule coded for the insulin protein, which produce strands of amino acids that are coded for an immature form of the biologically active insulin molecule referred to as ‘pro-insulin’. Once the pro-insulin molecule is generated it then undergoes modification by several enzymes including prohormone convertase one (PC1), prohormone convertase two (PC2) and carboxypeptidase E, which results in the production of a biologically active insulin molecule. Once the biologically active insulin protein is generated it is stored in a vacuole in the Beta cell to await being released into the blood stream.

Insulin receptors, which appear on the surface of cells, offer binding sites for insulin circulating in the blood. When insulin binds to an insulin receptor, the biologic response inside the cell causes glucose to enter the cell and undergo processing in the cytoplasm. Processed glucose molecules then enter the mitochondria. The mitochondria further process the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach to the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the Beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-γ), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in the United States. Especially in diabetic patients that are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from Beta cells as required. The deleterious effects of diabetes may still evolve despite strict and persistent control of the glucose level in the blood stream.

The current treatment of diabetes may be augmented by the unique approach to utilizing modified viruses as vehicles to transport quantum genes into cells in order to increase the production of biologically active insulin. By utilizing modified viruses to transport quantum genes to facilitate and enhance the production of mRNAs, which would then facilitate the assembly of proteins would offer a new treatment option for patients with diabetes.

Viruses are obligate parasites. Viruses simply represent a carrier of genetic material and by themselves viruses are unable to replicate or carry out any form of biologic function outside their host cell. Viruses are generally comprised of one or more nested shells constructed of one or more layers of protein or lipid material, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biochemical machinery for the virus to successfully replicate itself.

Protected by an outer protein coat or lipid envelope, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of their RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA (mRNA). RNA viruses that bypass the host cell's DNA, cause the cell to in general generate copies of the necessary parts of the virus directly from the virus's RNA genome.

Present medical care is attempting to utilize viruses to deliver genetic information into cells. Research in the field of gene therapy has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the outside wall of a virus virion dictate which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA payload to the cell type the viral vector naturally targets as its host cell.

Naturally occurring viruses also have the disadvantage of being susceptible to detection and elimination by a body's immune system. Viruses have been infecting humans for hundreds of thousands of years. A human's innate immune system is very efficient at detecting the presence of most naturally occurring viruses when such a virus is inside the body. The human immune system is quite capable of generating a vigorous response to most intruding viruses, attacking and neutralizing virus virions whenever a virus virion physically exists are outside the exterior wall of the virus's host cell. If gene therapy in its current state were to become a clinical therapeutic tool, the naturally occurring viruses selected for gene therapy research will have limited effectiveness due the fact that once the viral vector is introduced into the body, the body's the immune system will quickly engage and eliminate the viral vectors, possibly before the vector is able to deliver its payload to its host cell or target cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

Cichutek does not claim the use of specific glycogen probes to target specific types of cells. Cichutek's approach is dependent upon the probes naturally present on the viral vectors reported in the patent, which will direct the viral vectors to only those cells the viruses naturally use as their host cell. Cichutek's approach is very restrictive, limited to gene transfer to only cells the viruses use as their natural host cell.

Cichutek's claim 4, states The SIVagm vector of claim 1, wherein the viral envelope further comprises a single chain antibody (scFv) or a ligand of a cell surface molecule.' By use of the words ‘a’ and ‘or’ in the claim, the claim is limited in the singular, meaning Cichutek claims a single chain antibody or a singular ligand. Singular type antibodies or ligands can be used for cell to cell communication, but to open an access portal into a cell and insert a payload into the cell requires two different types antibodies or ligands. As an example human immunodeficiency virus requires the use of both the gp120 and gp41 probes to open a portal into a T-Helper cell and insert its viral genome into the T-Helper cell. The gp120 probe engages the CD4+ cell-surface receptor on the T-cell. Once the gp120 probe has successfully engaged a CD4+ cell-surface receptor on the target T-Helper cell, then the HIV virion's gp41 probe can engage either a CXCR4 or a CCR5 cell-surface receptor on the T-Helper cell in order to open up an access portal for HIV to insert its viral genome into a T-Helper cell. It is well documented in the medical literature that a genetic defect leading to an abnormality in the CXCR4 cell-surface receptor prevents HIV virions from opening an access portal and inserting its genetic payload into such T-Helper cells. This genetic defect in the CXCR4 cell-surface receptor offers the subset of people carrying the genetic defect resistance to HIV infection. This example demonstrates the need for at least two types of glycoprotein probes to be present on the surface of a viral vector in order for a viral vector to be capable of opening an access portal and delivering the payload the vector carries into its host cell or target cell.

A delivery system that offered a defined means of targeting specific types of cells would invoke minimal or no response by the innate immune system and the adaptable immune system when present in the body, and a delivery system that would be capable of inserting into cells a wide variety of quantum gene molecules would significantly improve the current medical treatment options available to clinicians treating patients.

The solution to arriving at a versatile, workable delivery system that will meet the needs of a number of medical treatments involves three important elements. These elements include:
(1) configurable external probes whereby more than one type of protein structure probe or more than one type of glycoprotein probe is to be used to engage and access specific target cell types in order to successfully deliver a payload into a specific type of cell,
(2) an exterior envelope comprised of a protein shell or lipid layer expressing the least number of cell-surface markers, such as the use of a stem cell to act as the host cell to manufacture the delivery devices,
(3) configuring the core of the vector to enable it to carry and deliver quantum genes.
   For purposes of this text, the use of the terms ‘specific target cell type’, ‘target cell’, ‘specific type of cell’, ‘specific cell’, ‘specific type of cell’ are equivalent and interchangeable; the configuration of cell-surface receptors that a specific type of cell has located on and protruding from its outer cell membrane determines the cell type.

Viruses are obligate parasites. Viruses simply represent a carrier of genetic material and by themselves viruses are unable to replicate or carry out any form of biologic function outside their host cell. A ‘virion’ refers to the physical structure of a single complete virus as it exists outside of the host cell; a more archaic term for ‘viral virion’ was ‘viral particle’. Viruses are generally comprised of one or more nested shells constructed of one or more layers of protein, some with a lipid outer envelope, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's external probes is to locate and engage a host cell's receptors. The virus's surface probes are designed to detect, make contact with and functionally engage one or more receptors located on the exterior of the type of cell that will offer the virus the proper environment in which to construct copies of itself. A host cell provides the virus the proper biologic machinery for the virus to successfully replicate itself. Once the virus's genome is inside the host cell, the viral genome takes command of the cell's production machinery and causes the host cell to generate copies of the virus. As the viral copies exit the host cell, these virions set off in search of other host cells to infect.

Naturally occurring viruses exist in a number of differing shapes. The shape of a virus may be rod or filament like, icosahedral, or complex structures combining filament and polygonal shapes. Viruses generally have their outer wall comprised of a protein coat or an envelope comprised of lipids.

An outer envelope comprised of lipids may be in the form of one or two phospholipid layers. When the outer envelope is comprised of two phospholipid layers this is termed a lipid bilayer. For purposes of this text the term ‘lipid’ includes ‘phospholipid’ molecules. A phospholipid is a composite molecule comprised of a polar or hydrophilic region on one end and a nonpolar or hydrophobic region on the opposite end. A lipid bilayer covering a virus, like the membrane of a cell, is constructed with the hydrophilic region of one of the phospholipid layers pointed toward the exterior of the virion and the hydrophilic region of the second phospholipid layer pointed inward toward the center of the virus virion; with the hydrophobic regions of each of the two lipid layers pointed toward each other. The outer envelope of some forms of virus may be comprised of an outer lipid layer or lipid bilayer affixed to a protein matrix for support, the protein matrix being located closer to the center of the virus virion than the lipid layer or lipid bilayer.

Spherical viruses are generally spherical in shape and may be comprised of an outer envelope and one inner shell or alternatively an outer envelope and multiple inner shells. Inner shells are approximately spherical in shape; this is because the proteins comprising the protein matrix shell have an irregular shape to their structure, but when constructed together for a shape that resembles a sphere. In the case of a spherical virus with an outer envelope and one inner shell, the inner shell is often referred to as a nucleocapsid shell comprised of numerous capsid proteins attached to each other. In the case of a spherical virus being comprised of an outer envelope and multiple inner shells, the outermost inner viral shells may be referred to as comprised of a quantity of matrix proteins, where the innermost shell is referred to as a nucleocapsid and is comprised of a quantity of capsid proteins. The inner protein shells are nested inside each other. The cavity created by the innermost shell or nucleocapsid is referred to as the ‘core’ or ‘center of the virus’. Any payload carried by the virus virion is generally carried in the core or center of the virion.

Viruses carry genetic material in the form of deoxyribonucleic acid (DNA) or ribonucleic acid (RNA) as their payload. DNA or RNA genome payloads are carried in the cavity of the nucleocapsid referred to as the core. A virus is therefore generally considered to be a DNA virus if its genome is comprised of DNA or the virus is considered a RNA virus if its genome is comprised of RNA that acts as genetic instructions to generate copies of the virus. Viruses may also carry enzymes as part of their payload. An enzyme such as ‘reverse transcriptase’ transforms a RNA viral genome into DNA. Protease enzymes modify the viral genome once it has entered a host cell. An integrase enzyme assists a DNA viral genome with insertion into the host cell's nuclear DNA. The entire genetic payload is carried inside the cavity created by the virus's nucleocapsid shell.

The probes attached to the exterior of a virus are constructed to engage specific cell-surface receptors on specific type of cells in the body. Only a cell that expresses cell-surface receptors that are capable of being engaged by the probes of a specific virus can act as a host for the virus. Viruses generally use two probes to access a host cell. The first probe makes an initial attachment to the host cell, while the action of the virus's second probe often in conjunction with the action of the first probe cause an access portal to be created in the host cell's exterior plasma membrane. Once an access portal is formed, the virus inserts the contents of its payload into the host cell utilizing the open access portal. Certain types of virus may be engulfed whole by a target cell. Once the virus's genome is inside the cytoplasm of the host cell, any enzymes that accompanied the viral genome into the cell, may begin to modify or assist the virus's genome with infecting and taking control of the host cell's biologic functions.

Probes are attached to the exterior envelope of a virus virion. Probes may be in the form of a protein structure or may be in the form of a glycoprotein molecule. For viruses constructed with a protein matrix as its outer envelope, the probes tend to be protein structures. A portion of the protein structure probe is fixed or anchored in the protein matrix, while a portion of the protein structure probe extends out and away from the protein matrix. The portion of the protein structure probe extending out away from the virus virion is referred to as the ‘exterior domain’, the portion anchored in the protein matrix is the ‘transcending domain’. Some protein probes have a third segment that extends through the envelope and exists inside the virus virion, which is referred to as the ‘interior domain’. The exterior domain of a protein structure probe is intended to engage a specific cell-surface receptor on a biologically active cell the virus is targeting as its host cell.

Viruses that utilize a lipid layer as the outer envelope, are constructed with probes that tend to be glycoproteins. A glycoprotein is comprised of a protein segment and a carbohydrate segment. The carbohydrate segment of the glycoprotein molecule is fixed or anchored in the lipid layer of the outer envelope, while the protein segment extends outward and away from the outer envelope. The protein portion of a glycoprotein probe that extends outward and away from the outer envelope of a virus virion is intended to engage a cell-surface receptor on a biologically active cell the virus is targeting as its host cell.

Some forms of viruses that utilize a lipid layer as its envelope use protein structure probes. In this case, the portion of the protein structure probe that extends outward and away from the outer envelope is the ‘exterior domain’, the portion that is anchored in the lipid layer is the ‘transcending domain’ and again some protein structure probes have an ‘interior domain’ that exist inside the virion, which may also help anchor the protein structure probe to the virion. The exterior domain of a protein structure probe that extends outward and away from the outer envelope of a virus virion is intended to engage a cell-surface receptor on a biologically active cell the virus is targeting as its host cell.

When a virus carries a DNA payload and the viral DNA is inserted into the host cell, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of the viral RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA. RNA viruses that bypass the host cell's DNA, cause the cell in general to generate copies of the necessary parts of the virus directly from the virus's RNA genome.

The human immunodeficiency virus (HIV) is a RNA virus and has an outer envelope comprised of a lipid bilayer. The lipid bilayer covers a protein matrix consisting of p17^{gag} proteins. Inside the p17^{gag} protein is nested a nucleocapsid comprised of p24^{gag} proteins. Inside the nucleocapsid HIV carries its payload. HIV's genetic payload consists of two single strands of RNA and several enzymes. The enzymes that accompany HIV's genome include ‘reverse transcriptase’, ‘integrase’ and ‘protease’ molecules.

The T-Helper cell acts as HIV's host cell. The HIV virion utilizes two types of glycoprotein probes affixed to its exterior envelope to locate and engage a T-Helper cell. HIV utilizes a glycoprotein probe 120 to locate a CD4 cell-surface receptor on a T-Helper cell. Once an HIV glycoprotein 120 probe has successfully engaged a CD4 cell surface-receptor on a T-Helper cell a conformational change occurs in the glycoprotein 120 probe and a glycoprotein 41 probe is exposed. The glycoprotein 41 probe's intent is to engage a CXCR4 or CCR5 cell-surface receptor on the same T-Helper cell. Once a glycoprotein 41 probe on the HIV virion successfully engages a CXCR4 or CCR5 cell-surface receptor, the HIV virion opens an access portal through the T-Helper cell's outer membrane.

Once the HIV virion has opened an access portal through the T-Helper cell's outer plasma membrane, the HIV virion inserts two positive strand RNA molecules and the associated enzymes it carries into the T-Helper cell. Each RNA strand is approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the HIV's DNA becomes inserted into the T-Helper cell's native nuclear DNA. When the timing is appropriate, the now integrated viral DNA is decoded by the host cell's polymerase molecules and the virus's genetic information commands certain cell functions to carry out the replication process to construct copies of the human immunodeficiency virus.

The outer layer of the HIV virion is comprised of a portion of the T-Helper cell's outer cell membrane. In the final stage of the replication process, as a copy of the HIV virion, carrying the HIV genome, buds through the host cell's cell membrane the outer protein shell acquires as its exterior envelope, a wrapping of lipid bilayer from the host cell's cell membrane. In the case of HIV, since the surface of the pathogen is covered by an envelope comprised of lipid bilayer taken from the host T-Helper cells, this feature allows the HIV virion the capacity to elude the two immune systems, since the detectors comprising the innate immune system and the adaptable immune system may find it difficult to distinguish between the surface of an infectious HIV virion and the surface characteristics of a noninfected T-Helper cell.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C viral genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus life-cycle demonstrates that copies of a virus virion can be generated by inserting RNA into a host cell that functions as messenger RNA in the host cell. The Hepatitis C viral RNA genome functions as messenger RNA, acting as the template in conjunction with the biologic machinery of a host cell to produce the components that comprise copies of the Hepatitis C virion and the Hepatitis C viral RNA provides the biologic instructions to assemble the components into complete copies of the Hepatitis C virions. The Hepatitis C virus life-cycle clearly demonstrates that viral virions can be manufactured by a host cell without involving the nucleus of the cell.

Deciphering the existence, replication and behavior of viruses provides clear examples of several fundamental concepts, which include: (1) Viruses target specific cells in the body by means of identifying and engaging such target cells utilizing the probes projecting outward from the virus's exterior shell to make contact with cell-surface receptors located on the surface of the target cells, and (2) Viruses are capable of carrying a variety of different types of payloads including DNA, RNA and a variety of proteins.

Current gene therapy approach to attempting to deliver a payload to cells in the body use modified forms of existing viruses to act as transport devices to deliver genetic information. This approach is severely limited by restricting the virus virion to the target only cells the viral vector naturally seeks out and infects. Current gene therapy approach is further limited by using the pre-existing size of naturally occurring viruses, rather than being able to modify the size of the structure to be able to tailor the volumetric carrying capacity of the payload portion of the modified virus. Further, gene therapy is restricted to utilizing naturally occurring viruses to deliver only genetic information; it has not previously been appreciated by those skilled in the art that virus-like transport devices might deliver to a variety of specific type of cells a wide variety of differing payloads such as quantum genes.

A dramatic, not previously recognized by those expert in the art is the need to develop a transport vehicle that can be fashioned to seek out specific types of cells and deliver to these cells DNA genetic material. The exterior envelope of a transport should be constructed so as not to alert the immune system of its presence to prevent rejection of the vehicles. Transport vehicles should be capable of being configured to target any specific type of cell and engage and deliver their payload only to that specific type of cell.

An equally dramatic, not previously recognized is the need for strict organization of the genes that comprise the human genome. The individual genes must each be labeled with some form of unique identifier to facilitate the nuclear transcription mechanisms in easily finding the transcribable genetic information when needed.

Merging these two concepts together suggests a transport device that would be capable of inserting quantum genes into specifically targeted cells; a constellation of concepts not previously before recognized by those skilled in the art.

For purposes of this text, the term ‘exogenous’ refers to an item which originates outside the boundaries of a particular cell or cell type and becomes a part of a particular cell or cell type. The term ‘endogenous’ refers to an item which originates as a part of a particular cell or cell type and remains a part of that particular cell or cell type.

### BRIEF SUMMARY OF THE INVENTION

Utilization of configurable microscopic medical payload delivery devices to deliver quantum genes to specific type of cells facilitates a dramatic new approach to medical care. By selecting the type of probes that are present on the surface of the configurable microscopic medical payload delivery devices, specific types of cells can be targeted. By delivering quantum genes to specific type of cells, genetic instructions delivered to cell can be located and transcribed in an efficient manner, and thus utilized in the specific type of cells in a timely fashion. A wide variety of medical conditions are manageable by utilizing this new and unique approach.

### DETAILED DESCRIPTION

The future of medical treatment will be the widespread utilization of a medical treatment delivery method the incorporates configurable microscopic medical payload delivery devices (CMMPDD) to deliver quantum genes directly to targeted cell types in the body.

Introduced herein is a delivery method that includes the concepts: (1) configurable microscopic medical payload delivery devices can carry quantum gene molecules as the payload, and (2) glycoprotein probes present on the exterior of the configurable microscopic medical payload delivery devices include specific glycoprotein probes or protein structure probes affixed to the exterior, these glycoprotein probes or protein structure probes intended to seek out and engage cell-surface receptors attached to the exterior of whichever cell the configurable microscopic medical payload delivery devices is intended to deliver its payload of quantum gene molecules in order to produce a predetermined medically beneficial effect.

For the purposes of this text a ‘quantum gene’ is comprised of a sequence of nucleotides that represents a ‘unique identifier’ physically linked to a sequence of nucleotides that represent a discrete quantity of genetic information; these sequences of nucleotides being comprised of some combination of the nucleotides being referred to by their nitrogenous base as adenine (A), thymine (T), cytosine (C), and guanine (G). The genetic information associated with the above-mentioned unique identifier may be comprised of a portion of transcribable genetic information and a portion of nontranscribable genetic information which together define a specific gene, otherwise referred to as a discrete quantity of genetic information. The nontranscribable segments of a quantum gene may represent segments that act as instructions such as a START code, STOP code and REPEAT code or may help facilitated the attachment of a transcription complex or be simply ignored during the transcription process. Quantum gene molecules can be comprised of a segment of nucleotides where the portion that represents a unique identifier is separated from the portion that represents genetic information by a quantity of base pairs of nucleotides that do not represent a unique identifier and do not represent genetic information. The purpose of the separation of the portion of the unique identifier from the portion of the genetic information by a quantity of base pairs of nucleotides that do not represent a unique identifier and does not represent genetic information is to facilitate a transcription complex attaching to the quantum gene upstream from the portion of the quantum gene that represents genetic information so that transcription of the biologic information associated with the quantum gene may occur.

The genetic information in a quantum gene codes for some combination of protein coding RNA (pcRNA), non-coding RNAs (ncRNA) and spacers. Spacers represent segments of the DNA that do not code for a RNA molecule. The genetic information in a quantum gene, when transcribed, produces protein coding RNA and non-coding RNA. Protein coding RNAs, usually referred to as messenger RNAs, undergo the process of translation in the cytoplasm of the cell and produce proteins. Non-coding RNAs are highly abundant and functionally important for the cell's operation. Non-coding RNAs have also been referred by such terms as non-protein-coding RNAs (npcRNA) or non-messenger RNA (nmRNA) or small non-messenger RNA (snmRNA) or functional RNAs (fRNA). The non-coding RNAs include: transfer RNAs (tRNA), ribosomal RNAs (rRNA), small nuclear RNAs (snRNA), small nucleolar RNAs (snoRNA), signal recognition particle RNA (SRP RNA), antisense RNA (aRNA), micro RNA (miRNA), small interfering RNA (siRNA), Y RNA, telomerase RNA.

Transfer RNAs (tRNA), are RNAs that carries amino acids and deliver them to a ribosome. Ribosomal RNAs (rRNA), are RNAs that couple with ribosomal proteins and participate in translation of mRNA to produce protein molecules. Small nuclear RNAs (snRNA) are RNAs involved in splicing and other nuclear functions. Small nucleolar RNAs (snoRNA) are RNAs involved in nucleotide modification. Signal recognition particle RNA (SRP RNA) are RNAs are involved in membrane integration. Antisense RNA (aRNA) are RNAs involved in transcription attenuation, mRNA degradation, mRNA stabilization, and translation blockage. Micro RNA (miRNA) are RNAs involved in gene regulation and have been implicated in a wide range of cell functions including cell growth, apoptosis, neuronal plasticity, and insulin secretion. Small interfering RNA (siRNA) are RNAs involved in gene regulation, often interfering with the expression of a single gene. Y RNA are RNAs involved in RNA processing and DNA replication. Telomerase RNA are RNAs involved in telomere synthesis.

In addition to the unique identifier, a quantum gene is comprised of the biologic instruction code, which when transcribed produces one or more of the same RNA molecules or different RNA molecules. A quantum gene must be comprised of a unique identifier and the genetic material to code for at least one RNA molecule. The definition of a ‘quantum gene’ differs from all previous definitions of a ‘gene’ due to the requirement that the quantum gene must have a unique identifier that accompanies a segment of genetic information. From a medical treatment perspective, the quantum gene's unique identifier allows the genetic information present in the quantum gene to be located by a cell's transcription machinery, once the quantum gene is inserted into a cell's nuclear DNA.

Ribonucleic acid molecules directly transcribed from the DNA or quantum gene, may be precursor ribonucleic acid molecules that require modification by nuclear enzymes prior to being translatable or may be ribonucleic acid molecules which are directly translatable without further modification.

In the DNA there are a number of nucleotides physically existing along the deoxyribonucleic acid between the unique identifier and the transcribable genetic information; or in other terms a number of nucleotides that are not a part of the identification code and are not transcribable, exist downstream from the unique identifier and upstream from the transcribable genetic information.

It is well recognized that within the transcribable genetic information there exist subsegments of nucleotides that are not transcribable and there are subsegments of nucleotides that are transcribed but are not found in the final version of the RNA molecule. Subsegments of transcribable genetic information that are not transcribed are subsegments such as ‘STOP’ codes, which indicate to the transcription complex a potential point at which to cease transcribing the genetic information. Certain factors may influence whether a transcription complex actually ceases transcription at that point or whether the transcription complex continues transcribing when the transcription complex reaches a ‘STOP’ code. Subsegments of nucleotides that are transcribed and appear in the final active form of a RNA are referred to as exons. Subsegments of nucleotides that are transcribed, but do not appear in the final active form of a RNA are referred to as introns. Precursor RNA molecules include both exons and introns. Introns are removed by modification of the initial RNA segment directly transcribed from the transcribable genetic information.

Utilization of the sigma summation symbol to show summation over a series of indexed variables or expression can be represented as:

Σⱼ₌₁ⁿ[K]ⱼ=[K]₁+[K]₂+ . . . +[K]ₙ

An equation to represent a quantum gene would be:

$$Quantum gene = [ unique identifier ] + {\sum }_{a = 0}^{n} {[ nontranscribable connector nucleotide ]}_{a} + {\sum }_{b = 1}^{n} {[ nucleotide segment transcribable for RNA ]}_{b} + {\sum }_{c = 0}^{n} {[ nontranscribable spacer nucleotide ]}_{c} + {\sum }_{d = 0}^{n} {[ nontranscribable nucleotide commands ]}_{d}$$

Where ‘unique identifier’ represents a number, a name or the combination of a number and a name that the transcription complex utilizes to locate a specific quantum gene amongst the DNA material present in a biologically active cell.
Where ‘nontranscribable connector nucleotide’ represents one or more nucleotides that physically exists between the ‘unique identifier’ and the segment of ‘transcribable genetic information’.
Where a ‘nontranscribable spacer nucleotide’ represents one or more nucleotides comprising the transcribable genetic information that is not transcribed when the transcription complex transcribes the genetic information of the quantum gene.
Where a ‘nontranscribable nucleotide command’ represents one or more nucleotides comprising the transcribable genetic information that is not transcribed when the transcription complex transcribes the genetic information of the quantum gene, but acts as an instruction to the transcription complex to cause the transcription complex to function in a certain manner; examples include a STOP code that causes the transcription complex to cease transcription and a REPEAT code that causes the transcription complex to repeat its transcription of a segment of genetic material.
Where ‘a’ represents the range of ‘zero to any positive whole number.
Where ‘b’ represents the range of ‘one to any positive whole number’.
Where ‘c’ represents the range of ‘zero to any positive whole number’.
Where ‘d’ represents the range of ‘zero to any positive whole number’.
Where the DNA segment that is transcribable for RNA may transcribe RNAs that may exist in a precursor form; such a precursor form may include elements such as introns that are removed following transcription by modifying proteins.

For purposes of this text an ‘external envelope’ refers to the outermost covering of a virus or a virus-like transport device or a configurable microscopic medical payload delivery device. The external envelope may be comprised of a lipid layer, a lipid bilayer, the combination of a lipid layer affixed to a protein matrix or the combination of a lipid bilayer affixed to a protein matrix. A protein matrix is equivalent to a protein shell and may be referred to as a protein matrix shell. The terms protein matrix, protein shell, protein matrix shell are equivalent to the term capsid, where the term capsid is meant to represent ‘a protein coat or shell of a virus particle, surrounding the nucleic acid or nucleoprotein core’. For purposes of this text, the term ‘particle’ is equivalent to the term ‘virion’; further the term ‘virus particle’ is equivalent to ‘viral virion’.

For purposes of this text an ‘internal shell’ refers to a protein matrix shell nested inside the external envelope. Multiple inner shells may exist, with those of smaller diameter concentrically nested inside those of a larger diameter. The inner most protein matrix shell is termed the nucleocapsid. The proteins that comprise the nucleocapsid are termed capsid proteins. In the cavity created by the nucleocapsid, referred to as the center or core of the nucleocapsid, is where the payload of quantum gene molecules is carried.

For purposes of this text ‘external probes’ are molecular structures that are utilized to locate and engage cell-surface receptors on biologically active cells. External probes are generally comprised of a portion which is anchored or fixed in the external envelope and a second portion that extends out and away from the external envelope. The portion of the external probe that extends out and away from the external envelope is intended to make contact and engage a specific cell-surface receptor located on a biologically active cell. External probes may be comprised solely of a protein structure or an external probe may be a glycoprotein molecule.

For purposes of this text ‘glycoprotein molecule’ refers to a molecule comprised of a carbohydrate region and a protein region. Glycoprotein molecules that act as probes are generally anchored or fixed to a lipid layer utilizing the carbohydrate portion of the molecule as an anchor. The protein portion of the glycoprotein molecule which extends outward and away from the exterior envelope the glycoprotein has been affixed such that the protein region may function as a probe to locate and attach to the cell-surface receptor it was created to engage.

The concept of configurable microscopic medical payload delivery devices is modeled after naturally existing viruses. Configurable microscopic medical payload delivery devices in general are spherical in shape; though other shapes may be used as function might warrant the use of a particular shape. The spherical configurable microscopic medical payload delivery devices are comprised of an exterior envelope and one or more inner nested protein shells. A quantity of exterior protein structure probes and/or glycoprotein probes are anchored in the exterior envelope and a portion extend out and away from the exterior lipid envelope. Nesting of protein shells refers to progressively smaller diameter shells fitting snugly inside protein shells of a larger diameter. Inside the inner most protein shell, referred to as the nucleocapsid, is a cavity referred to as the core of the device. The core of the device is the space where the medically therapeutic payload the device carries is located. The payload of the device is comprised of ribonucleic acid molecules.

Configurable microscopic medical payload delivery devices (CMMPDD) target specific types of cells in the body. Configurable microscopic medical payload delivery devices engage specific types of cells by the configuration of probes affixed to the exterior envelope of the CMMPDD. By fixing specific probes to the exterior envelope of the CMMPDD, these probes intended to engage and attach only to specific cell-surface receptors located on certain cell types in the body, the CMMPDD will deliver its payload only to those cell types that express compatible and engagable specific cell-surface receptors. In a similar fashion where the exterior probes of a naturally occurring virus engage specific cell-surface receptors present on the surface of the virus's host cell and only the designated host cell, the CMMPDD's exterior probes are configured to engage cell-surface receptors on a specific type of target cell and only those cells. In this manner, the payload of quantum gene molecules carried by CMMPDD will be delivered only to specific types of cells in the body. The configuration of exterior probes on the surface of a CMMPDD varies as needed so as to effect the CMMPDD delivery of specific quantum gene payloads to specific type of cells as needed to effect a particular predetermined medical treatment.

The size of the configurable microscopic medical payload delivery devices is dependent upon the diameter of the inner protein matrix shells and this is dictated by the volume size of the payload the CMMPDD is required to carry and deliver to a target cell. The diameter of the each inner protein matrix shell is governed by the number of protein molecules utilized to construct the protein matrix shell at the time the protein matrix shell is generated. Increasing the number of proteins that comprise a protein matrix shell increases the diameter of the protein matrix shell. When applicable, as dictated by the capacity the CMMPDD is to be utilized to function as, an external lipid envelope wraps around and covers the outer protein matrix shell. The larger the volume of the core of the CMMPDD, the greater the physical size of the payload the CMMPDD is able to carry. The size of the configurable microscopic medical payload delivery device is to be generally the size of cell (approximately 10⁻⁴ m in diameter) or less, generally detectable by a light microscope or, as needed, an electron microscope. The size of the CMMPDD is not to be too large such that it would generate a burden to the body by damaging organ tissues through clogging blood vessels or the glomeruli in the kidneys. The dimensions of each type of CMMPDD are to be tailored to the mission of the CMMPDD, which takes into account factors such as the type of target cell, the size of the payload that is to be delivered to the target cells and the length of time the CMMPDD may engage the target cell.

Being enveloped in an external lipid layer, configurable microscopic medical payload delivery devices possess the advantage of having their exterior appear similar to the plasma membrane that acts as an outside covering for the cells that comprise the body. By appearing similar to existing plasma membranes, the CMMPDDs appear similar to naturally occurring structures found in the body. CMMPDD are afforded the capability to avoid detection by a body's immune system because the exterior of the CMMPDD mimics the cells comprising the body and the surveillance elements of the immune system find it difficult to discern between the CMMPDD and naturally occurring cells comprising the body.

To carry out the process of manufacturing a configurable microscopic medical payload delivery device, a primitive cell such as a stem cell is selected. The reason for utilizing primitive cells such as stems cells as the host cell, is that the CMMPDD acquires its outer envelope from the host cell and the more primitive the host cell, the fewer in number the identifying protein markers are present on the surface of the CMMPDD. The fewer the identifying surface proteins present on the outer envelope of the CMMPDD, the less likely a body's immune system will identify the CMMPDD as an invader and therefore less likely the body's immune system will react to the presence of the CMMPDD and reject the CMMPDD by attacking and neutralizing the CMMPDD.

Stem cells used as host cells to manufacture quantities of CMMPDD product are selected per histocompatibility markers present on their surface. Certain histocompatibility markers present on the surface of the final CMMPDD product will be less likely to cause a reaction in a specific patient based on the genetic profile of the patient's histocompatibility markers. A similar histocompatibility match is done when donor organs are selected to be given to recipients to avoid rejection of the donor organ by the recipient's immune system.

The selected stem cell used to manufacture configurable microscopic medical payload delivery devices goes through several steps of maturation before it is capable of generating therapeutic CMMPDD product. RNA inserted into the host stem cell code for the general physical outer structures of the CMMPDD. RNA inserted into the host generate surface probes that target the cell-surface receptors on a specific target cell type. RNA would be inserted into the host that would be used to generate the payload of quantum genes. Similar to how copies of a naturally occurring virus, such as the Hepatitis C virus or HIV, are produced, assembled and released from a host cell, copies of the CMMPDD would be produced, assembled and released from a stem cell functioning as a de facto host cell. Once released from the host cell, the copies of the CMMPDD would be collected, then pooled together to produce a therapeutic dose that would result in a medically beneficial effect.

The stem cells used as host cells are suspended in a broth of nutrients and are kept at an optimum temperature to govern the rate of production of the CMMPDD product. Similar to the natural production of the Hepatitis C virus, the configurable microscopic medical payload delivery devices ‘production genome’ is introduced into the host stem cells. The configurable microscopic medical payload delivery devices production genome carries genetic instructions to cause the host cells to manufacture the configurable microscopic medical payload delivery devices' outer protein wall, the inner protein matrixes, the surface probes the configurable microscopic medical payload delivery device is to have affixed to its outer envelope and the quantity of quantum gene molecules the configurable microscopic medical payload delivery devices are to carry; and the instructions to assemble the various pieces into the final form of the configurable microscopic medical payload delivery devices and the instructions to activate the budding process. The resultant configurable microscopic medical payload delivery devices are collected from the nutrient broth surrounding the host cells and placed together into doses to be used as a treatment for a medical disease.

The ‘production genome’ are an array of RNAs, which include messenger RNAs that are directly translated by the host cell's ribosomes. The production genome dictates the characteristics of the final version of the CMMPDD that buds from the host stem cell and is released and is to be utilized as a medical treatment. The production genome is specifically tailored to code for the surface probes that will seek and engage a specific type of target cell. The production genome also carries the instructions to code for the production of the type of quantum genes to be delivered to the specific type of target cell. The ‘production genome’ varies depending upon the configuration of the CMMPDD and the specific type of quantum genes the CMMPDD will transport to effect a specific medical treatment on a specific type of cell.

The configurable microscopic medical payload delivery device transporting quantum gene molecules represents a very versatile medical treatment delivery device. CMMPDD is used to deliver a number of different quantum gene molecules to a wide variety of cells in the body.

The construction of a naturally occurring virus can be likened to the act of following a programmed script to produce a specific result. It is known that the genetic code that a virus carries dictates the production of copies of the virus. It is known that specific segments of the viral genetic code represent instructions that dictate the construction of different parts of the virus so that copies of the virus can be made inside the host cell. It is well documented that there exist different subtypes of most viruses, based off of mutations that have occurred to the viral genome over time; these mutations to the viral genome producing variants in the construction of the virus. Configurable microscopic medical payload delivery devices which carry quantum genes are constructed much like a naturally occurring virus virions would be constructed in a host cell. Altering the production RNA alters the configuration of the external probes or alters the configuration of the size of the inner shells or alters the type of quantum gene the CMMPDD will carry or alters any combination of the three.

As an example of the method to produce a device to treat diabetes mellitus utilizing configurable microscopic medical payload delivery devices to deliver to Beta cells quantum genes, which when transcribed produce messenger RNA coded to produce insulin, the following production process is followed in the lab: (1) human stem cells are selected. (2) Into the selected stem cells is placed the RNA production genome constructed, in this case, specifically as a means to treat diabetes mellitus. The RNA production genome contains genetic instructions to cause the host stem cells to manufacture the CMMPDDs' outer protein wall, the inner protein matrix, surface probes to include a quantity of glycoprotein probes that engage the GPR40 cell-surface receptor present on the surface of Beta cells located in the Islets of Langerhans in the pancreas, and the payload of quantum genes, in this case the quantum genes to facilitate the production of the insulin molecules in Beta cells; and the biologic instructions to assemble the components into the final form of the CMMPDD; and the biologic instructions to activate the budding process. (3) Upon insertion of the RNA production genome into the host stem cells, host stem cells' production cellular machinery responds by simultaneously translating the different segments of the RNA production genome to produce the proteins that comprise the exterior protein wall, the inner protein matrix molecules, the surface probes, the quantum gene payload to produce the messenger RNA that will produce insulin, and decode the instructions to assemble the components into the CMMPDDs. (4) Upon assembly, the CMMPDDs bud through the cell membrane of the host stem cell. (5) At the time of the budding process, the CMMPDDs acquire an outside envelope wrapped over the outer protein shell, this outer envelope comprised of a portion of the plasma membrane from the host stem cell as the CMMPDDs exit the host cell. (6) The resultant CMMPDDs are collected from the nutrient broth surrounding the host stem cells. (7) The CMMPDD product is washed in sterile solution to separate the CMMPDD product from any unwanted elements of the nutrient broth. (8) The configurable microscopic medical payload delivery devices are removed from the sterile solvent and suspended in a hypoallergenic liquid medium. (9) The configurable microscopic medical payload delivery devices are separated into individual quantities to facilitate storage and delivery to physicians and patients. (10) The configurable microscopic medical payload delivery devices transported in the hypoallergenic liquid medium is administered to a diabetic patient per injection in a dose that is tailored to receiving patient's requirement to produce sufficient amount of insulin to control the blood sugar. (11) Upon being injected into the body, the configurable microscopic medical payload delivery devices migrate to the Beta cells located in the Islets of Langerhans by means of the patient's blood stream. (12) Upon the configurable microscopic medical payload delivery devices reaching the Beta cells, the configurable microscopic medical payload delivery devices engage the cell-surface receptors located on the Beta cells and insert the payload of quantum genes they carry into the Beta cells. The payload of quantum genes migrate to the nucleus of the Beta cells. The quantum genes inserts into the nuclear DNA of the Beta cells. Transcription machinery present in the nucleus transcribes the quantum genes. Messenger RNAs generated by transcribing the exogenous quantum genes enhances the Beta cells' production of insulin molecules. The increase in insulin production by Beta cells successfully manages diabetes mellitus.

In a similar fashion, configurable microscopic medical payload delivery devices can be fashioned to deliver a payload of a specific type of quantum gene molecule to any type of cell in the body. Different cell types express different cell-surface markers on the exterior of their plasma membrane. The differing configurations of cell-surface markers on differing types of cells distinguish one cell type from another cell type. By configuring the exterior probes that extend from the surface of the configurable microscopic medical payload delivery device to seek out and engage specific cell-surface receptors present on a specific type of cell, payloads of any quantum gene molecule can be delivered to specific cells in the body.

The transcribable genetic information linked to the unique identifier may occur in the form of naturally found transcribable genetic information or may occur as artificially created transcribable genetic information, referred to as ‘artificial transcribable genetic information’. Naturally found transcribable genetic information would be a segment of transcribable genetic information that would be found in a cell's genome otherwise referred to as a gene. Artificial transcribable genetic information would be transcribable genetic information that would represent either (i) a modified form of a naturally occurring gene or (ii) a segment of nucleotides that represents transcribable genetic information that is artificially created to produce a medically beneficial result.

A quantum gene, as it exists as a functional part of the deoxyribonucleic acid of a cell, is a segment of deoxyribonucleic acid, comprised of both a unique identifier and a segment of transcribable biologic information, that is capable of being inserted into a cell's nuclear DNA. DNA is comprised of two parallel strands of nucleotides. Each strand of DNA is a mirror image of each other since adenine must combine with thymine and cytosine must combine with guanine. Therefore, since each strand of DNA is a mirror image of each other, one strand of DNA possesses the nucleotide sequence that codes for both strands; one strand represents the DNA code, while the second strand represents the mirror image of the first strand. In this manner, a quantum gene can be defined in its most elemental form as a sequence of nucleotides comprising a single strand of nucleotides.

A quantum gene could thus be represented as a single strand of nucleotides comprised of the nucleotides adenine, cytosine, guanine and thymine. The double stranded form of a quantum gene would be the single strand of nucleotides attached in parallel to a second strand of nucleotides that represents the mirror image of the single strand of nucleotides. Double stranded deoxyribonucleic acid segments is the form quantum genes take when a quantum gene is inserted into a cell's nuclear genome.

### Conclusions, Ramification, and Scope

Accordingly, the reader will see that the method to utilize configurable microscopic medical payload delivery device to deliver quantum genes to specific targeted cell types provides advantages over existing art by (1) being a method to use delivery devices that seeks out specific types of cells, (2) by being method that uses delivery devices that are versatile enough to deliver a variety of quantum genes to accomplish various medical treatments and (3) by being a method to use delivery devices constructed with a surface envelope that will avoid detection by the innate as well as the adaptable immune systems so as not to activate the immune system to its presence; for these reasons this represents a new and unique medical delivery device that has never before been recognized nor appreciated by those skilled in the art.

The reader will also see that the concept and utilization of a method that incorporates the use of the quantum gene as described in this text has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

NUMBER OF DRAWINGS: 0

What is claimed:

1. A method for inserting quantum gene molecules into specific types of cells comprising:
(a) providing a quantity of protein shells,
(b) covering said protein shells with an exterior envelope,
(c) fixing a quantity of exterior probes to said exterior envelope, and
(d) positioning a quantity of quantum gene molecules inside the cavity created by the innermost said protein shell,
whereby said quantity of protein shells covered with said exterior envelope with said quantity of exterior probes affixed to said exterior envelope, with said quantum gene molecules carried in said cavity created by said inner most protein shell, engages said specific types of cells and inserts said quantum gene molecules into said specific types of cells,
whereby said exterior probes are intended to engage specific cell-surface receptors on said specific type of cell,
whereby said interior shells are versatile enough in their construction to carry within said cavity created by said innermost said protein shell a wide variety of quantum gene molecules to said specific type of cells,
whereby inserting said quantum gene molecules into said specific types of cells facilitates the successful management of protein deficient states inside said specific types of cells.

2. The method for inserting quantum gene molecules into specific types of cells in claim 1 wherein said external envelope is comprised of a quantity of lipid layers and a quantity of protein matrix shells.

3. The quantity of lipid layers in claim 2 wherein said quantity of lipid layers is a quantity of phospholipid layers.

4. The method for inserting quantum gene molecules into specific types of cells in claim 1 wherein said protein shells is comprised of a quantity of nested sphere-like concentric protein matrix shells.

5. The method for inserting quantum gene molecules into specific types of cells in claim 1 wherein said exterior probes are comprised of a quantity of protein structure probes and a quantity of glycoprotein probes.

6. The protein structure probes in claim 5 wherein said protein structure probes are comprised of a segment of said protein structure probes which extends outward and away from said exterior envelope, attached to a segment of said protein structure probe which is embedded in said exterior envelope,
whereby said segment of said protein structure probes which extends outward and away from said exterior envelope is intended to engage said specific cell-surface receptors on said specific type of cell,
whereby said segment of protein structure probe embedded in said exterior envelope is intended to hold the said protein structure probe affixed to said external envelope.

7. The protein structure probes in claim 5 wherein said protein structure probes are comprised of a plurality of protein structure probes,
whereby, at least two differing configurations of said protein structure probes may be needed to successfully engage said specific type of cell with one type of said configuration of said protein structure probe engaging one type of said specific cell-surface receptor, while a differing type of said configuration of said protein structure probe is required to engage a differing type of said specific cell-surface receptor in order for said configurable microscopic medical payload delivery device to insert said quantity of quantum gene molecules said configurable microscopic medical payload delivery device carries into intended said specific type of cell.

8. The glycoprotein probes in claim 5 wherein said glycoprotein probes are comprised of a protein segment, which extends outward and away from said exterior envelope, which is attached to a carbohydrate segment, said carbohydrate segment being embedded in said exterior envelope,
whereby said protein segment which extends outward and away from said exterior envelope is intended to engage said specific cell-surface receptor on said specific type of cell,
whereby said carbohydrate segment embedded in said exterior envelope is intended to hold the said glycoprotein probe affixed to said external envelope.

9. The glycoprotein probes in claim 5 wherein said glycoprotein probes are comprised of a plurality of glycoprotein probes,
whereby, at least two differing configurations of said glycoprotein probes may be needed to successfully engage said specific type of cell with one type of said configuration of said glycoprotein probe engaging one type of said specific cell-surface receptor, while a differing type of said configuration of said glycoprotein probe is required to engage a differing type of said specific cell-surface receptor in order for said configurable microscopic medical payload delivery device to insert said quantity of quantum gene molecules into said specific type of cell.

10. The method for inserting quantum gene molecules into specific types of cells in claim 1 wherein said quantum gene is comprised of a quantity of nucleotides which represent a unique identifier and a quantity of nucleotides which represent transcribable genetic information.

11. The unique identifier in claim 10 wherein said unique identifier is a unique sequence of nucleotides which represents a unique identifier for said transcribable genetic information.

12. The unique identifier in claim 10 wherein said unique identifier is physically connected to said transcribable genetic information, along a nucleotide strand, said unique identifier positioned on the side of said transcribable genetic information where a transcription complex assembles along said nucleotide strand and begins to transcribe said transcribable genetic information.

13. The unique identifier in claim 10 wherein said unique identifier is a segment of nucleotides comprised of a unique array of nucleotides that represent a naturally occurring unique identifier of said transcribable genetic information or represents an artificial unique identifier of said transcribable genetic information,
whereby naturally occurring genes already have a unique identifier that can be used for a transcription complex to locate said naturally occurring gene once an exogenously produced said naturally occurring gene is inserted into a cell's nuclear genome,
whereby artificially created genes would require an artificial unique identifier in order for a transcription complex to locate said artificial gene once said exogenously produced artificial gene was inserted into a cell's nuclear genome.

14. The transcribable genetic information in claim 10 wherein said transcribable genetic information is comprised of genetic code that when transcribed by a cell's transcription complex produces a quantity of ribonucleic acid molecules.

15. The ribonucleic acid molecules in claim 14 wherein said ribonucleic acid molecules may be precursor ribonucleic acid molecules that require modification by nuclear enzymes prior to being translatable or may be ribonucleic acid molecules that are directly translatable without further modification.

16. The method for inserting quantum gene molecules into specific types of cells in claim 1 wherein said quantum gene is comprised of said segment of nucleotides comprised of said portion which represents said unique identifier physically attached to, but separated from said portion which represent said transcribable genetic information by a quantity of nucleotides that do not represent said unique identifier and do not represent said transcribable genetic information.

17. The transcribable genetic information in claim 10 wherein said transcribable genetic information when transcribed produces a quantity of precursor ribonucleic acid molecules that require modification by enzymes in order to become functional ribonucleic acid molecules or said transcribable genetic information when transcribed produces a quantity of ribonucleic acid molecules that are fully functional when said transcribable genetic information is transcribed.

18. The transcribable genetic information in claim 10 wherein said transcribable genetic information when transcribed produces a quantity of ribonucleic acid molecules found in nature and produces a quantity of ribonucleic acid molecules not found in nature, but which are artificially created to perform a medically beneficial function.

19. The quantity of nucleotides in claim 10 wherein said quantity of nucleotides are comprised of a quantity of adenine nucleotides, a quantity of cytosine nucleotides, a quantity of guanine nucleotides, and a quantity of thymine nucleotides.

20. The quantity of nucleotides in claim 1 wherein said quantity of nucleotides is a segment of deoxyribonucleic acid.
